(19) United States
(12) Patent Application Publication
(10) Pub. No.: US 2010/0316740 A1
(43) Pub. Date: Dec. 16, 2010

(54) COMPOSITIONS FOR THE TREATMENT OF BENIGN PROSTATE HYPERTROPHY, PROSTATITIS, PROSTATOSIS AND PROSTATE CANCER

(75) Inventor: Ezio Bombardelli, Groppello Cairoli (PV) (IT)

Correspondence Address: YOUNG & THOMPSON, 209 Madison Street, Suite 500, Alexandria, VA, 22314, US

(73) Assignee: INDENA S.P.A., Milano (IT)

(21) Appl. No.: 12/918,393

(22) PCT Filed: Feb. 17, 2009
(86) PCT No.: PCT/EP09/01080

§ 371 Date: Aug. 19, 2010

(30) Foreign Application Priority Data
Feb. 22, 2008 (IT) MI2008A000283

(51) Int. Cl. A61K 9/02; A61K 36/889; A61P 35/00; A61P 13/08; A61M 31/00

(52) U.S. Cl. 424/727; 604/288

(57) ABSTRACT

Compositions including Serenoa repens, Echinacea and Hypericum perforatum extracts and selenium compounds for the treatment of prostate disorders, in particular for the treatment of prostate cancer, prostatitis, prostatosis and urinary incontinence.

### SUMMARY OF THE INVENTION

The present invention relates to compositions comprising Serenoa repens, Echinacea and Hypericum perforatum extracts and selenium compounds for the treatment of prostate disorders, in particular for the treatment of prostate cancer, prostatitis, prostatosis and urinary incontinence.

The compositions according to the invention exert an anti-inflammatory action which is useful in the treatment of prostatitis and prostatosis, and in the treatment and prevention of prostate cancer deriving from degeneration of said conditions.

Moreover, said compositions are useful for the treatment of non-bacterial prostatitis with an inflammatory component and the pelvic pain generally associated with it, which frequently occurs in numerous patients even in the absence of full-blown disease.

### PRIOR ART

The male aging process is associated with an increased frequency of benign, and often malignant, alterations of the prostate gland. These conditions reflect uncontrolled growth of the components of the stroma and epithelia of the gland. Autopsies performed on the prostates of men aged 70-80 have demonstrated hyperplastic changes in 90% of individuals, with malignant degenerations in 70% of cases; in the majority of the cases these alterations were not diagnosed during the patient's lifetime.

Consequently, any treatment must be carefully evaluated in the patient, to ensure that no problems exceeding the natural morbidity are created.

Medical and surgical treatments, particularly in the benign proliferative disorder, characterised by the classic symptoms of pollakiuria, dysuria and obstruction, must take account of the side effects which can ensue if the treatment is performed too early in adult life.

In the case of tumours, the evaluation of the risk/benefit ratio must take account of the patient's quality of life.

The incidence of prostate tumours and the resulting mortality has declined in recent years, despite the dramatic increase in the number of cases diagnosed with the use of markers (PSA), for reasons which are not yet fully understood. This negative trend has probably been influenced by the use of unconventional drugs and a more correct diet. In any event, prostate tumours are still the most common type of tumour in men, and represent the second-highest cause of death. Many tumours are asymptomatic in the early stages, whereas benign hyperplastic proliferations cause particularly unpleasant symptoms, which are easily detected by the patient. If these symptoms are not suitably treated, by treating the causes as far as possible, they may degenerate, and in the case of urine retention may give rise to recurrent infections and serious health problems. The bladder also contributes to the symptoms of prostate disorders; emptying of the bladder is not only associated with prostate hypertrophy, but also with modifications of the trigone in which the cannabinoid and vanilloid receptors are involved.

It has been reported in the recent literature that prostate cancer, like benign prostate hypertrophy, is often associated with an altered ratio between the oestrogens and androgens that govern the normal prostate functions. In addition to problems associated with bacterial infections which are also one of the causes, an altered hormonal metabolism often produces local inflammatory processes which become chronic, degenerating into malignant tumoral forms. Treatment of these disorders requires the administration of anti-inflammatories, antibiotics and antiandrogens for long periods, and the results are not always satisfactory. Long-term use of antibiotics considerably reduces the body's defenses, with unforeseeable consequences on the proliferation of any tumour cells which may already be present.

Pharmaceutical or para-pharmaceutical preparations which promote the preventive rather than curative aspect of many symptoms of the urinary apparatus can be very useful, provided that they are well-tolerated and non-toxic.

### DESCRIPTION OF THE INVENTION

The Applicant has found that a combination comprising lipophilic extracts of Serenoa repens, Echinacea and Hypericum perforatum, and selenium compounds, is particularly effective for the prevention and treatment of prostate disorders, especially prostate cancer, prostatitis, prostatosis and urinary incontinence.

The present invention therefore relates to compositions comprising a combination of:
a) lipophilic extract of Serenoa repens,
b) lipophilic extract of Echinacea,
c) a component selected from lipophilic extract of Hypericum perforatum, hydrogenated lipophilic extract of Hypericum perforatum and octahydrohyperforin,
d) selenium compounds,
   for the prevention and treatment of prostate disorders.

More particularly, the present invention relates to compositions comprising a combination of:
a) lipophilic extract of Serenoa repens,
b) lipophilic extract of Echinacea or the isobutylamides contained therein,
c) a component selected from lipophilic extract of Hypericum perforatum, hydrogenated lipophilic extract of Hypericum perforatum and octahydrohyperforin or hyperforin derivatives,
d) a selenium compound selected from methylselenocysteine and a selenide.

Serenoa repens extract controls prostate hypertrophy and reduces symptoms such as dysuria, pollakiuria and urine retention, due to its aromatase-inhibiting and dihydrotestosterone receptor binding activity. Serenoa repens extract is also known for its effect on prostate growth factors (b-FGF, VFGF and insulin growth factor), which helps to reduce the progress of the disorder. However, despite its interesting action mechanism, this extract alone is not very active on the progression of hypertrophy and is practically inactive against prostatitis and prostatosis of various etiologies due to the lack of a marked anti-inflammatory and antibacterial action.

The effect of Serenoa repens extract on the prostate is boosted by the isobutylamides present in the lipophilic extract of Echinacea, especially E. angustifolia, which also reduce dihydrotestosterone binding to the androgen receptors (Endocrinology, 1980, 107, 848-50), thus reducing hyperplastic stimulation.

The isobutylamides present in the lipophilic extract of Echinacea inhibit cyclooxygenase-2, preventing the formation of PGE₂ at the site of inflammation (Biochemical and Biophysical Res. Comm. 2007), and therefore have a marked anti-inflammatory and analgesic effect, which is useful to control hypertrophy. These substances are mainly eliminated through the renal emunctory, and have a particular tropism for the prostate and the bladder, where they bind to the cannabinoid receptors, which are abundantly present. These compounds are consequently useful to combat urinary incontinence, especially in woman. Moreover, the cannabinoid receptor ligands are considered useful to reduce the risk of malignant degeneration due to cell transformation into anaplastic and neoplastic cells. The isobutylamides are also significant immunomodulating agents, which is particularly important in reducing inflammation and reinfection of the prostate.

The hydrogenated lipophilic extract of Hypericum perforatum is characterised by the presence of hyperforin derivatives, especially octahydrohyperforin. Octahydrohyperforin is an antimicrobial agent towards numerous Gram +ve bacteria, and also has a potent anti-inflammatory action and mild antidepressant effect, which is useful in obtaining the patient's compliance. Octahydrohyperforin is a lipophilic substance which is well absorbed in humans, ensuring appreciable plasma levels and justifying its therapeutic use in prostate hypertrophy and inflammatory states.

For reasons which are not yet understood, selenium boosts the activity of many drugs, including those with anti-tumoral activity. The presence of selenium derivatives in the combination strengthens the effect of the other components. In particular, selenomethylcysteine possesses cell protection and tumour prevention properties, including prevention of prostate tumours.

The combination according to the invention has proved to possess a surprisingly marked effect of reducing inflammatory states and cell proliferation in the prostate. This combination can therefore be used effectively to treat prostate disorders, including particularly resistant forms of prostatitis. The global effect in the prostate of the compositions according to the invention can be attributed to the synergic effect of the combination, not to a single component.

According to a preferred aspect, the compositions according to the invention will contain the various components within the following weight intervals:
a) lipophilic extract of Serenoa repens: 100 to 350 mg,
b) lipophilic extract of Echinacea: 2 to 200 mg,
c) lipophilic or hydrogenated lipophilic extract of Hypericum perforatum: corresponding to 10 to 100 mg of octahydrohyperforin,
d) methylselenocysteine: 50 to 150 μg.

According to a particularly preferred aspect, the compositions will contain the various components in the following weight amounts:
a) lipophilic extract of Serenoa repens: 200 mg,
b) lipophilic extract of Echinacea angustifolia: 10 mg,
c) hydrogenated lipophilic extract of Hypericum perforatum: corresponding to 50 mg of octahydrohyperforin,
d) methylselenocysteine: 100 μg.

According to a preferred aspect, the compositions of the invention will contain a lipophilic extract of Echinacea angustifolia, pallida or purpurea. According to an even more preferred aspect, the compositions of the invention will contain a lipophilic extract of Echinacea angustifola containing approx. 30% isobutylamides, prepared according to the process disclosed in EP 464298.

The compositions according to the invention will be formulated according to conventional techniques for the formulation of lipophilic ingredients for the oral or rectal administration. Examples of formulations are soft gelatin capsules or cellulose capsules suitable to contain oily substances, or rectal pessaries or suppositories. The rectal route, using suppositories that slowly release the active components, has proved particularly convenient.

These formulations will be administered once or twice a day, preferably twice a day at the start of the treatment, and once a day, preferably in the mornings, for maintenance treatment.

The reduction in the symptoms of prostate hypertrophy begins by the second day, and no side effects have been recorded in placebo-controlled trials. In prostatosis, the effect begins more slowly; after the first week the inflammatory plasma parameters are modified, an increase in the immunological parameters is observed which is useful to combat chronic inflammation, and organ distension improves. Long-term treatment with the compositions according to the invention leads to surprising prevention of the prostatosic degeneration of the inflamed prostate due to correlated bacterial infections.

According to a further aspect, the compositions of the invention can be administered in concomitance with other substances having a useful or complementary activity, such as specific antibiotics or anti-inflammatory agents with particular prostate or bladder tropism.

The following examples will help to illustrate the invention.

### EXAMPLE I

### Capsules

Each capsule contains:

| | | |
|---|---|---|
| Hydrogenated lipophilic extract of Hypericum perforatum | 50 | mg |
| Lipophilic extract of Echinacea angustifolia | 10 | mg |
| Methylselenocysteine | 100 | μg |
| Lipophilic extract of Serenoa repens | q.s. to 350 | mg |

### EXAMPLE II

### Capsules

Each capsule contains:

| | | |
|---|---|---|
| Octahydrohyperforin salt of dicyclohexylamine | 20 | mg |
| Lipophilic extract of Echinacea angustifolia | 20 | mg |
| Methylselenocysteine | 100 | μg |
| Lipophilic extract of Serenoa repens | q.s. to 350 | mg |

### EXAMPLE III

### Suppositories

| | | |
|---|---|---|
| Hydrogenated lipophilic extract of Hypericum | 75 | mg |
| perforatum | | |
| Lipophilic extract of Echinacea angustifolia | 25 | mg |
| Methylselenocysteine | 125 | μg |
| Lipophilic extract of Serenoa repens | q.s. to 200 | mg |
| Semisynthetic glycerides | q.s. to 2500 | mg |

1-10. (canceled)

11. A composition comprising:
a) lipophilic extract of Serenoa repens;
b) lipophilic extract of Echinacea;
c) a component selected from the group consisting of lipophilic extract of Hypericum perforatum, hydrogenated lipophilic extract of Hypericum perforatum and octahydrohyperforin; and
d) selenium compounds.

12. The composition as claimed in claim 11, comprising:
a) lipophilic extract of Serenoa repens,
b) lipophilic extract of Echinacea or the isobutylamides contained therein,
c) a compound selected from the group consisting of hydrogenated lipophilic extract of Hypericum perforatum, derivatives of hyperforin and octahydrohyperforin,
d) a selenium compound selected from methylselenocysteine and a selenide.

13. The composition as claimed in claim 11, comprising:
a) 100 to 350 mg of lipophilic extract of Serenoa repens;
b) 2 to 200 mg of lipophilic extract of Echinacea;
c) an amount of hydrogenated lipophilic extract of Hypericum perforatum: corresponding to 10 to 100 mg of octahydrohyperforin; and
d) 50 to 150 mg of methylselenocysteine.

14. The composition as claimed in claim 13, comprising:
a) 200 mg of lipophilic extract of Serenoa repens;
b) 10 mg of lipophilic extract of Echinacea angustifolia;
c) an amount of lipophilic extract or hydrogenated lipophilic extract of Hypericum perforatum corresponding to 50 mg of octahydrohyperforin; and
d) 100 mg of methylselenocysteine.

15. The composition as claimed in claim 11, wherein the lipophilic extract of Echinacea is selected from the group consisting of Echinacea angustifolia, Echinacea pallida and Echinacea purpurea.

16. The composition as claimed in claim 15, wherein the lipophilic extract of Echinacea is Echinacea angustifola with 30% isobutylamides.

17. The composition as claimed in claim 11, for the oral or rectal administration.

18. The composition as claimed in claim 17, wherein the composition is in a form selected from the group consisting of soft gelatin capsules, cellulose capsules suitable to contain oily substances, rectal pessaries and suppositories.

19. A method of treating prostate disorders, comprising administering to a subject in need thereof an effective amount of a composition comprising:
a) lipophilic extract of Serenoa repens,
b) lipophilic extract of Echinacea or the isobutylamides contained therein,
c) a compound selected from the group consisting of a lipophilic extract Hypericum perforatum, a hydrogenated lipophilic extract of Hypericum perforatum, derivatives of hyperforin, and octahydrohyperforin,
d) a selenium compound selected from methylselenocysteine and a selenide.

20. The method as claimed in claim 19, wherein the prostate disorders are selected from group consisting of prostate cancer, prostatitis, prostatosis and urinary incontinence.
